(19) United States
(12) Patent Application Publication
(10) Pub. No.: US 2025/0026032 A1
(43) Pub. Date: Jan. 23, 2025

(54) FLEXURAL STRUCTURE AND HALF-FINISHED PRODUCT THEREOF

(71) Applicant: NHK SPRING CO., LTD., Kanagawa (JP)

(72) Inventors: Takafumi HIRATA, Kanagawa (JP); Yuki HOTODA, Kanagawa (JP); Yuki HAYAKAWA, Kanagawa (JP)

(73) Assignee: NHK SPRING CO., LTD., Kanagawa (JP)

(21) Appl. No.: 18/713,179

(22) PCT Filed: Nov. 25, 2022
(86) PCT No.: PCT/JP2022/043500

§ 371 Date: May 24, 2024

(30) Foreign Application Priority Data
Nov. 26, 2021 (JP) 2021-192500

(51) Int. Cl. B25J 18/06; A61B 34/30

(52) U.S. Cl. CPC B25J 18/06; A61B 34/30

(57) ABSTRACT

A flexural structure, in which a multi-structured flexible body is capable of displacing from an extended state to a flexed state, includes: an outer coil spring, constituting the multi-structured flexible body; an inner coil spring, provided inside the outer coil spring and having a wire overlap a wire of the outer coil spring in a coil radial direction to constitute the multi-structured flexible body; a one-side receiving member, receiving one side of the multi-structured flexible body; and an other-side receiving member, receiving an other side of the multi-structured flexible body. A close-contact state between wires of the inner and outer coil springs and is set in accordance with a spacing between the one-side receiving member and the other-side receiving member.

### TECHNICAL FIELD

The present invention relates to a flexural structure to be used in a robot, a manipulator, or the like, and a half-finished product thereof.

### RELATED ART

Some robots, manipulators, actuators, and the like are provided with a flexural structure that enables bending and extension. Such a flexural structure is disclosed in, for example, Patent Literature 1.

The flexural structure of Patent Literature 1 has a double coil structure, enabling bending and extension. Such a flexural structure is formed by screwing the inner coil part into the inner periphery of the outer coil part while the outer coil part is stretched, and the wires of the outer coil part and the inner coil part are in close contact with each other.

As a result, the flexural structure is in a state in which an initial tension (a force that constantly tries to bring the coil wires into close contact with each other even when there is no load) is applied. Since this initial tension depends on the structure of the outer coil part and the inner coil part, in order to set a different initial tension, it is necessary to change the structure of the outer coil part and the inner coil part, which is complicated.

### CITATION LIST

### Patent Literature

[Patent Literature 1] Japanese Patent Application Publication No. 2020-26021.

### SUMMARY OF INVENTION

### Technical Problem

The problem to be solved is the complicated setting of different initial tensions.

### Solution to Problem

The present invention is a flexural structure, in which a multi-structured flexible body is capable of displacing from an extended state to a flexed state, the flexural structure including: an outer coil spring, constituting the multi-structured flexible body: an inner coil spring, provided inside the outer coil spring and having a wire overlap a wire of the outer coil spring in a coil radial direction to constitute the multi-structured flexible body: a one-side receiving member, receiving one side of the multi-structured flexible body; and an other-side receiving member, receiving an other side of the multi-structured flexible body. The flexural structure is provided such that a close-contact state between wires of the inner coil spring and the outer coil spring is set in an extended state of the multi-structured flexible body in accordance with a spacing between the one-side receiving member and the other-side receiving member.

Further, the present invention is a half-finished product of a flexural structure in which a multi-structured flexible body is capable of displacing from an extended state to a flexed state, the half-finished product of the flexural structure including: an outer coil spring, constituting the multi-structured flexible body: an inner coil spring, provided inside the outer coil spring and having a wire overlap a wire of the outer coil spring in a non-close-contact state in a coil radial direction to constitute the multi-structured flexible body; and a one-side receiving member, receiving one side of the multi-structured flexible body. The half-finished product of the flexural structure is provided such that when assembled to a device side, a close-contact state between wires of the inner coil spring and the outer coil spring is set in an extended state of the multi-structured flexible body in accordance with a spacing between the one-side receiving member and an other-side receiving member on the device side.

### Effects of Invention

According to the present invention, different initial tensions may be easily set by setting the close-contact state between the wires of the inner coil spring and the outer coil spring in accordance with the spacing between the one-side receiving member and the other-side receiving member.

### BRIEF DESCRIPTION OF DRAWINGS

FIG. 1 is a perspective view showing the main part of the manipulator to which the flexural structure according to Embodiment 1 of the present invention is applied.

FIG. 2 is a cross-sectional view of the manipulator of FIG. 1.

FIG. 3 is a schematic diagram showing the main part of FIG. 2.

FIGS. 4(A) and (B) are conceptual diagrams of the manipulator of FIG. 1, in which FIG. 4(A) shows the state before the initial tension is loaded, and FIG. 4(B) shows the state after the initial tension is loaded.

FIGS. 5(A) and (B) are conceptual diagrams of the manipulator of FIG. 1, in which FIG. 5(A) shows the extended state, and FIG. 5(B) shows the flexed state.

FIGS. 6(A) and (B) are comparisons of the return characteristics of the flexural structure from the flexed state to the extended state with the comparative examples, in which FIG. 6(A) is a graph showing overall changes, and FIG. 6(B) is an enlarged graph of the vicinity of the origin of FIG. 6(A).

FIGS. 7(A) and (B) are conceptual diagrams of the manipulator to which the flexural structure according to Embodiment 2 of the present invention is applied, in which FIG. 7(A) shows the extended state, and FIG. 7(B) shows the flexed state.

FIG. 8 is a conceptual diagram showing the manipulator to which the flexural structure according to Embodiment 3 of the present invention is applied.

FIG. 9 is a conceptual diagram showing the manipulator to which the flexural structure according to Embodiment 4 of the present invention is applied.

### DESCRIPTION OF EMBODIMENTS

The present invention achieves the object of easily setting different initial tensions by setting a close-contact state between wires of an inner coil spring and an outer coil spring in accordance with a spacing between a one-side receiving member and an other-side receiving member.

That is, the flexural structure 1 has a multi-structured flexible body 15 that is bendable from an extended state to a flexed state and includes an outer coil spring 23, an inner coil spring 25, a one-side receiving member 13, and an other-side receiving member 14.

The outer coil spring 23 and the inner coil spring 25 constitute the multi-structured flexible body 15. The inner coil spring 25 is provided inside the outer coil spring 23 and has a wire overlap a wire of the outer coil spring 23 in a coil radial direction. The one-side receiving member 13 receives one side of the multi-structured flexible body 15, while the other-side receiving member 14 receives an other side of the multi-structured flexible body 15. In the multi-structured flexible body 15, the close-contact state between the wires of the inner and outer coil springs 23 is set in accordance with a spacing between the one-side receiving member 13 and the other-side receiving member 14.

When setting the close-contact state, a friction between the wires may be set to zero or infinitesimally small with respect to a restoring force of the multi-structured flexible body 15 returning from the flexed state to the extended state.

The flexural structure 1 may include a close-contact setting part 21 that adjusts the spacing between the one-side receiving member 13 and the other-side receiving member 14 and setting a close-contact state between the wires of the inner coil spring 25 and the outer coil spring 23.

The close-contact setting part 21 is only required to be able to set the close-contact state between the wires of the inner and outer coil springs 23, and the structure may be freely set and realized according to the application target, etc.

The flexural structure 1 may include a plurality of cord-like members 19, one side of which is fixed to the one-side receiving member 13 and an other side of which is routed through the other-side receiving member 14, for operating a displacement of the multi-structured flexible body 15 to the flexed state, and the close-contact setting part 21 may share the cord-like members 19 when setting the close-contact state.

Further, the flexural structure 1 may include an operation part 22 that is provided on the other side of the cord-like members 19 and that operates a displacement between the extended state and the flexed state of the multi-structured flexible body 15 by relative pulling and pulling back operations between the plurality of cord-like members 19, the close-contact setting part 21 may support the operation part 22 in a position adjustable manner toward a direction of the multi-structured flexible body 15 in the extended state, and the close-contact state may be set by adjusting a position of the operation part 22 in the extended state of the multi-structured flexible body 15.

The operation part 22 may include a pulley 29 that is supported by the other-side receiving member 14 and is rotatably operable and movable for wrapping the cord-like members 19 around the one-side receiving member 13, and the close-contact setting part 21 may include a tensioner 31 that adjusts a position of the pulley 29.

The close-contact setting part 21 may include receiving parts 33 provided on the other side of the cord-like members 19 and elastic bodies 35 interposed between the other-side receiving member 14 and the receiving parts 33 and applying tension to the plurality of cord-like members 19 with respect to the one-side receiving member 13 in an extended state of the multi-structured flexible body 15. The close-contact state may be set by applying the tension by the elastic bodies 35.

### Embodiment 1

### [Manipulator]

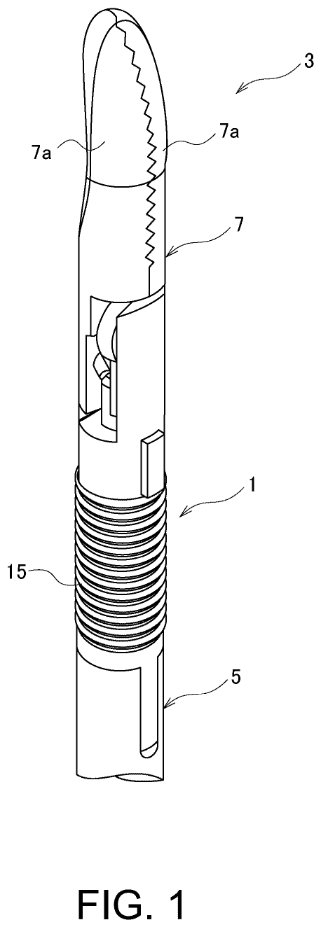

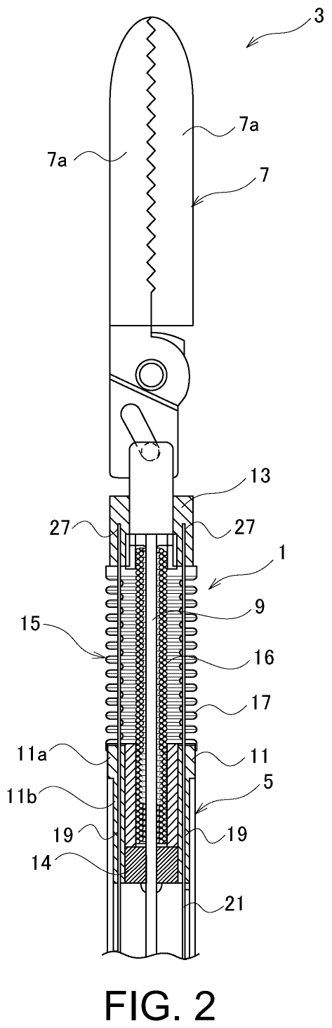

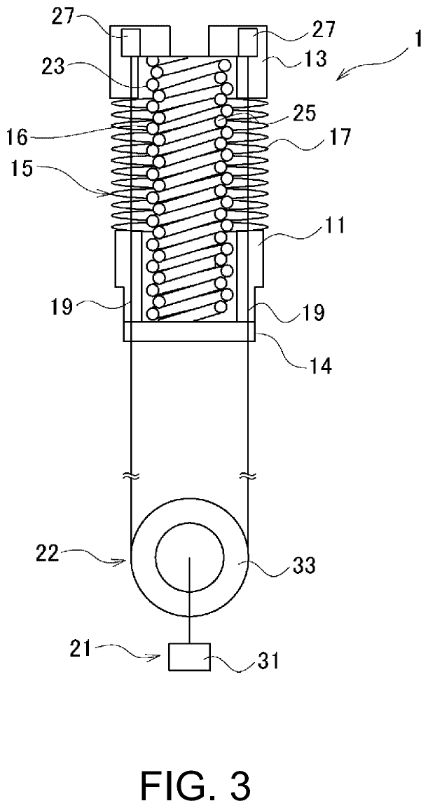

FIG. 1 is a perspective view showing the main part of the manipulator according to Embodiment 1 of the present invention, FIG. 2 is a cross-sectional view thereof, and FIG. 3 is a schematic diagram showing the main part of FIG. 2.

In this embodiment, a manipulator 3 using the flexural structure 1 will be described as an example. The manipulator 3 is medical forceps, and is used as forceps attached to a surgical robot, as well as endoscopes (cameras) not attached to a surgical robot, manual forceps, etc. The flexural structure 1 may be applied to a robot, a manipulator, an actuator, or the like, and may be applied to anything that requires bending.

The manipulator 3 includes a shaft 5, a flexural structure 1, and an end effector 7.

The shaft 5 is, for example, a cylindrical member. The end effector 7 is supported on the tip side of the shaft 5 via the flexural structure 1. The flexural structure 1 will be described later.

The end effector 7 is medical forceps, and a pair of gripping parts 7a are pivotally supported so as to be openable and closable with respect to a movable part 13 of the flexural structure 1, which will be described later. The end effector 7 is connected to a push-pull cable 9 that passes through the center of the shaft 5 and the flexural structure 1. The gripping part 7a is configured to open and close by the axial movement (advance and retreat) of the push-pull cable 9. In addition, when simply referring to an axial direction, it means a direction along the axis of the flexural structure 1 and includes a direction strictly parallel to the axis as well as a direction slightly inclined.

The gripping part 7a may be driven by air or the like. Further, the end effector 7 may be other than forceps, such as scissors, a grasping retractor, a needle driver, and the like.

### [Flexural Structure]

The flexural structure 1 includes an attaching base part 11, a movable part 13, a fixed receiving body 14, a multi-structured flexible body 15, driving wires 19, and a close-contact setting part 21. This flexural structure 1 is bendable between an extended state and a flexed state. It is noted that in this embodiment, the movable part 13 is the one-side receiving member, and the fixed receiving body 14 is the other-side receiving member.

The attaching base part 11 is configured to penetrate a double coil spring 16 as a multiple coil spring constituting the multi-structured flexible body 15 and to receive one end of a flexible tube 17. The attaching base part 11 includes a fitting part 11a and a head part 11b, and is a columnar body, particularly a stepped cylindrical body, made of resin, metal, or the like. The fitting part 11a of the attaching base part 11 is fitted and attached to the end tip of the shaft 5, and the head part 11b abuts against the edge of the shaft 5.

The attaching base part 11 is required to be able to receive one end of the flexible tube 17 and be coupled to the end part of the shaft 5, and the material, shape, and structure thereof may be freely set in accordance with the device to which the flexural structure 1 is applied.

The fixed receiving body 14 receives the other end of the double coil spring 16 and is fitted into and fixed to the end part of the shaft 5. The fixed receiving body 14 may be fixed to the shaft 5 by other techniques such as welding.

The fixed receiving body 14 is attached to the fitting part 11a of the attaching base part 11 in the shaft 5 in the axial direction. In addition, the fixed receiving body 14 may be provided with a gap in the axial direction with respect to the fitting part 11a. The fixed receiving body 14 may also be provided integrally with the shaft 5.

The movable part 13 is a columnar body, particularly a cylindrical body, made of resin, metal, or the like. The end effector 7 is attached to the movable part 13.

It is noted that the movable part 13 is not limited to a columnar body but may be a plate-like body or the like, as long as it is a member to which the end effector 7 may be attached. Further, the movable part 13 may be in an appropriate form in accordance with the device to which the flexural structure 1 is applied.

The movable part 13 is coupled to the attaching base part 11 by the multi-structured flexible body 15. As a result, the movable part 13 constitutes the one-side receiving member that receives one side of the multi-structured flexible body 15. That is, the other end of the flexible tube 17 is attached to the movable part 13, which receives one side of the double coil spring 16.

The multi-structured flexible body 15 is provided between the attaching base part 11, the fixed receiving body 14 and the movable part 13 in this embodiment. The multi-structured flexible body 15 is configured to be bendable between a flexed state and an extended state with respect to the axial direction.

This multi-structured flexible body 15 allows the movable part 13 to be displaced to a bending position and an extended position with respect to the attaching base part 11. The bending position is the position where the axis of the movable part 13 intersects with the axial direction and the bending of the flexural structure 1 is maximum. The extended position is the position where the axis of the movable part 13 is aligned with the axial direction. In the extended position, the axis of the movable part 13 does not need to be strictly aligned with the axial direction, and may be slightly deviated.

The multi-structured flexible body 15 of this embodiment includes the double coil spring 16 and the flexible tube 17. However, the flexible tube 17 may be omitted. Further, instead of the double coil spring 16, a multiple coil spring having three or more coils may be used.

The double coil spring 16 is a double coil that is bendable in the axial direction and includes an outer coil spring 23 and an inner coil spring 25.

The outer coil spring 23 and the inner coil spring 25 are each a compression coil spring having a predetermined gap (pitch) between the wires. The outer coil spring 23 and the inner coil spring 25 are compressed between the attaching base part 11 and the fixed receiving body 14 with the inner coil spring 25 located inside the outer coil spring 23.

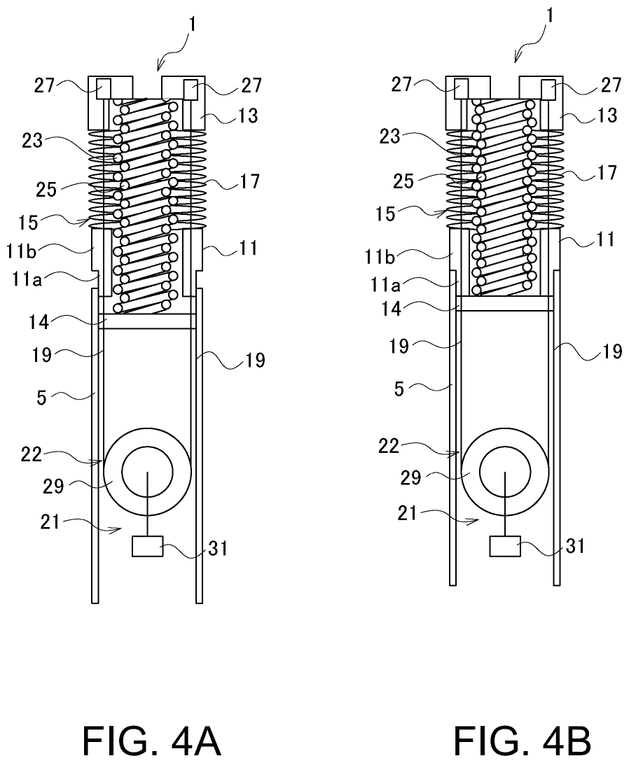

As a result, the inner coil spring 25 is screwed onto the inside of the outer coil spring 23. That is, the outer coil spring 23 and the inner coil spring 25 set a close-contact state between the wires in the extended state of the multi-structured flexible body 15 in accordance with the spacing between the attaching base part 11 and the fixed receiving body 14. In addition, the wires of the inner coil spring 25 and the outer coil spring 23 are in a non-contact manner in a free state (see FIG. 4(A)).

By this compression, a pressure contact force is applied to the double coil spring 16. The pressure contact force is a force that constantly causes the wires of the inner and outer coil springs 25 and 23 to come into close contact with each other.

In this embodiment, the initial tension is set in the close-contact state so that the friction between the wires is small with respect to the restoring force of the multi-structured flexible body 15 returning from the flexed state to the extended state. In particular, in this embodiment, the friction between the wires is set to be close to zero in the close-contact state.

In this embodiment, by setting the restoring force of the multi-structured flexible body 15 and the friction between the wires, the flexural structure 1 is allowed to be restored from the flexed state to the extended state. To this extent, there is no need to set the friction to zero.

However, the initial tension may be appropriately set by the close-contact setting part 21 in accordance with the device to which the flexural structure 1 is applied.

The inner coil spring 25 has a smaller coil diameter than the outer coil spring 23 and is provided so as to be screwed onto the inside of the outer coil spring 23. The coil diameters of the outer coil spring 23 and the inner coil spring 25 are constant from one end to the other end in the axial direction. However, the coil diameter of the outer coil spring 23 may also be changed in the axial direction.

The outer coil spring 23 has a plurality of gaps spaced in the axial direction between adjacent turns in the axial direction. The turns of the inner coil spring 25 fit into these gaps from the inside. Due to this fitting, the wire of the inner coil spring 25 has its outer circumferential part overlapping the wire of the outer coil spring 23 in the coil radial direction.

It is noted that the outer coil spring 23 and the inner coil spring 25 may be made of a material such as metal or resin. Further, the cross-sectional shape of the wires of the outer coil spring 23 and the inner coil spring 25 is circular. However, the cross-sectional shape of the outer coil spring 23 and the inner coil spring 25 is not limited to circular, and may be a rectangle, an ellipse, or the like. Further, the diameters of the wires of the outer coil spring 23 and the inner coil spring 25 may be different.

The flexible tube 17 constitutes the outer circumferential part of the multi-structured flexible body 15. Thus, the flexible tube 17 encloses the double coil spring 16. One end part of the flexible tube 17 is coupled to the movable part 13, and the other end part of the flexible tube 17 is coupled to the attaching base part 11.

The flexible tube 17 of this embodiment is made of accordion type bellows. However, the flexible tube 17 may be made of a plurality of wave washers stacked and coupled together by welding or the like, or may be made of a coil spring or other cylindrical body. In other words, the flexible tube 17 is not particularly limited as long as it has an elastic tubular shape.

The multi-structured flexible body 15 of this embodiment has a restoring force for returning from the flexed state to the extended state due to the cooperative elasticity of the flexible tube 17 and the double coil spring 16. When the flexible tube 17 is omitted, the restoring force is due to the elasticity of the double coil spring 16. It is noted that, in the case where the flexible tube 17 is provided, the double coil spring 16 may be configured so as not to provide the restoring force as the multi-structured flexible body 15.

The bending of the multi-structured flexible body 15 is performed by the driving wires 19. The driving wires 19 are cord-like members made of metal or the like, and are provided at four locations, spaced at spacings of 90°, in the circumferential direction of the flexural structure 1 in this embodiment. The driving wires 19 facing the radial direction of the flexural structure 1 form a pair. Thus, in this embodiment, two pairs of driving wires 19 are provided.

However, it is possible to omit one of the pair of driving wires 19, and the flexural structure 1 is only required to be provided with a plurality of driving wires 19. For example, three driving wires 19 may be provided. In this case, it is preferable that the driving wires 19 are provided at spacings of 120° in the circumferential direction. These driving wires 19 may be twisted wires, NiTi (nickel titanium) solid wires, piano wires, multi-joint rods, chains, strings, threads, ropes, or the like, so long as it is a cord-like member.

These driving wires 19 bend the flexural structure 1 when pulled in the axial direction, and are directly or indirectly connected to an operating mechanism (not shown) so as to be operated in the axial direction.

It is noted that the operation in the axial direction means pulling and pulling back the pair of driving wires 19 relative to each other in the axial direction. In this embodiment, when one of the pair of driving wire 19 is pulled in the direction of the attaching base part 11 with respect to the movable part 13, the other is pulled back toward the direction of the movable part 13. The driving wires 19 may be configured to be pulled independently of each other.

One side of each of the pair of driving wires 19 is a fixed part 27 fixed to the movable part 13. In addition, any fixing means may be applied to the fixed parts 27.

Each driving wire 19 extends from the fixed part 27 along the axial direction, passes through the flexible tube 17, the attaching base part 11, and the fixed receiving body 14, and has the other side routed through the inside of the shaft 5.

Each of the pair of driving wires 19 is connected via the pulley 29.

The pulley 29 is supported within the shaft 5 on the fixed receiving body 14 side. The pulley 29 is linked to an operating mechanism (not shown) and may be operated. The driving wires 19 are pulled and pulled back by operating the pulley 29. The pulley 29 may also be supported by an operating mechanism or the like outside the shaft 5. It is noted that the pulley 29 may be omitted and each driving wire 19 may be coupled to the operating mechanism.

The close-contact setting part 21 adjusts the spacing between the movable part 13 and the fixed receiving body 14 in the extended state of the multi-structured flexible body 15 and compresses the double coil spring 16 to set the close-contact state between the inner and outer coil springs 25 and 23. In this way, the initial tension of the double coil spring 16 is adjusted or set.

Further, in this embodiment, basically, the initial tension may be set simply by using the double coil spring 16 to attach the attaching base part 11 to the shaft 5. Thus, when there is not required to adjust the spacing between the movable part 13 and the fixed receiving body 14, the close-contact setting part 21 may be omitted. In this case, the omission is to omit the tensioner 31 to be described later.

The close-contact setting part 21 of this embodiment includes the driving wires 19 and the pulley 29 as the plurality of cord-like members. As described above, the driving wires 19 are configured such that one side is fixed to the movable part 13 and the other side is routed through the fixed receiving body 14 to operate the displacement of the multi-structured flexible body 15 to the flexed state. It is noted that the fixed receiving body 14 is only required to penetrate the other side of the driving wires 19 through a recess or a through hole.

The close-contact setting part 21 shares these driving wires 19 when setting the close-contact state between the wires of the inner and outer coil springs 25 and 23. Thus, the structure may be simplified.

The pulley 29 is supported in a position adjustable manner toward the axial direction of the multi-structured flexible body in the extended state, and By adjusting the position of the pulley 29 of the operation part 22 in the extended state of the multi-structured flexible body 15, the close-contact state between the wires of the inner and outer coil springs 25 and 23 is set.

When the position of the pulley 29 is adjusted, the relationship between the pulley 29 and the operating mechanism is also adjusted. In the structure in which the pulley 29 is supported by the operating mechanism, the position of the operating mechanism may be supported in a position adjustable manner. When the pulley 29 is omitted and each driving wire 19 is coupled to the operating mechanism, the position of the operating mechanism may be adjustable.

The close-contact setting part 21 is provided with a tensioner 31 that adjusts the position of the pulley 29. The tensioner 31 adjusts the position of the pulley 29 to set the close-contact state between the wires of the inner and outer coil springs 25 and 23.

### [Setting of Close-Contact State]

FIGS. 4(A) and (B) are conceptual diagrams of the manipulator of FIG. 1, in which FIG. 4(A) shows the state before the initial tension is loaded, and FIG. 4(B) shows the state after the initial tension is loaded.

As shown in FIG. 4(A) and FIG. 4(B), a half-finished product of the flexural structure 1 before application of initial tension is attached to the end part of the shaft 5, and an initial tension is applied to the double coil spring 16.

The half-finished product of the flexural structure 1 includes an outer coil spring 23, an inner coil spring 25, and a movable part 13, as shown in FIG. 4(A). In this half-finished product of the flexural structure 1, the inner coil spring 25 and the outer coil spring 23 of the double coil spring 16 are sparse, and there is a gap between the wires in the axial direction.

That is, the wires of the inner coil spring 25 and the outer coil spring 23 are in a non-close-contact state, with the wire of the inner coil spring 25 overlapping the outer coil spring 23 in the coil radial direction. It is noted that, in the half-finished product of the flexural structure 1, the wires of the inner coil spring 25 and the outer coil spring 23 may be in close contact with each other.

In this state, the end part of the double coil spring 16 penetrating the attaching base part 11 is abutted with the fixed receiving body 14, and the fitting part 11a of the attaching base part 11 is coupled to the end part of the shaft 5. As a result, the spacing between the movable part 13 and the fixed receiving body 14 is shortened, and the double coil spring 16 is compressed.

As a result, as shown in FIG. 4(B), the double coil spring 16 is compressed in accordance with the spacing between the movable part 13 and the fixed receiving body 14, and the wires of the inner coil spring 25 and the outer coil spring 23 are brought into close contact with each other. As a result, an initial tension is applied to the double coil spring 16.

It is noted that, in the half-finished product of the flexural structure 1, when the wires of the inner coil spring 25 and the outer coil spring 23 are in close contact with each other, an initial tension is applied and adjusted by compressing the double coil spring 16 corresponding to the spacing between the movable part 13 and the fixed receiving body 14.

In the case where further adjustment of the initial tension is required in this state, the spacing between the fixed receiving body 14 and the movable part 13 is adjusted in the extended state of the double coil spring 16 by the close-contact setting part 21. In other words, the position of the movable part 13 is adjusted by attracting the pulley 29 with the tensioner 31. This adjustment further compresses the double coil spring 16 and adjusts the close-contact state between the wires of the inner and outer coil springs 25 and 23.

Thus, in this embodiment, an initial tension may be applied to the double coil spring 16 with high accuracy. When the initial tension is to be adjusted beyond the settable range of the close-contact setting part 21, the position of the fixed receiving body 14 may be changed.

It is noted that when the attaching base part 11 abuts with the fixed receiving body 14, the wires of the inner and outer coil springs 25 and 23 are about to come into close contact with each other, and from this state, the position of the pulley 29 may be further adjusted to set the close-contact state.

Also, there may be a gap between the attaching base part 11 and the fixed receiving body 14, and the position of the pulley 29 may be adjusted to bring the wires of the inner and outer coil springs 25 and 23 closer together.

In this manner, in this embodiment, the close-contact state between the wires of the inner coil spring 25 and the outer coil spring 23 may be set in accordance with the spacing between the movable part 13 and the fixed receiving body 14, and different initial tensions may be easily set.

### [Operation]

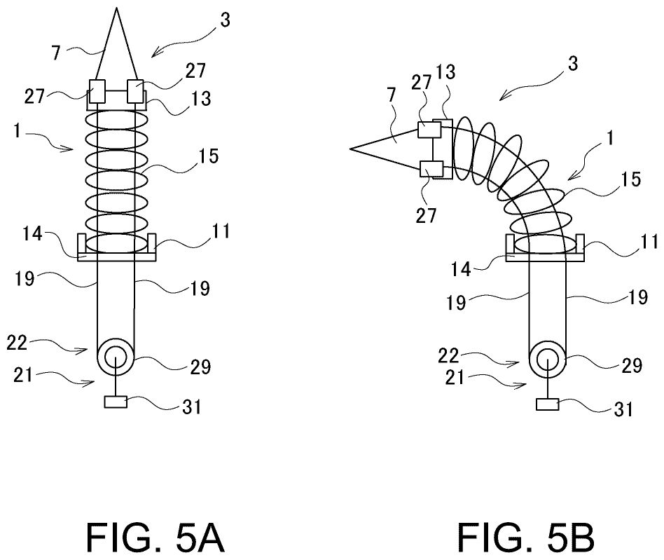

FIGS. 5(A) and (B) are conceptual diagrams of the manipulator of FIG. 1, in which FIG. 5(A) shows the extended state, and FIG. 5(B) shows the flexed state.

When an operator such as a doctor operates the manipulator 3, the flexural structure 1 is bent by pulling any one of the driving wires 19. The flexural structure 1 may be bent in all directions by 360° by combining and pulling different pairs of driving wires 19. In this way, the end effector 7 may be oriented in a desired direction.

After the flexural structure 1 is bent, the friction between the wires of the inner coil spring 25 and the outer coil spring 23 is set to be smaller than the restoring force of the double coil spring 16, so that the flexural structure 1 may be reliably restored to the original shape thereof.

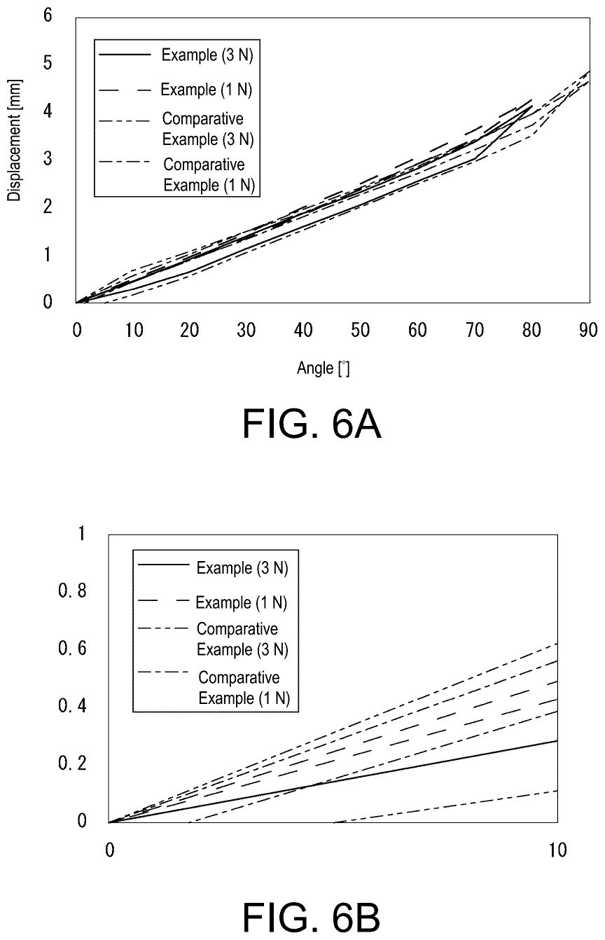

FIGS. 6(A) and (B) are comparisons of the return characteristics of the flexural structure from the flexed state to the extended state with the comparative examples, in which FIG. 6(A) is a graph showing overall changes, and FIG. 6(B) is an enlarged graph of the vicinity of the origin of FIG. 6(A).

In FIG. 6, Example (1N) is an example in which, as in FIG. 4(A), a load of 1N per driving wire 19 was applied in the axial direction to the double coil spring 16 of the example in which there was a gap between the wires, and the close-contact load of the wires was almost zero and the wires were in close-contact state. Example (3N) is an example in which a load of 3N per driving wire 19 was applied in the axial direction to the double coil spring 16 of the example in which the wires were in the close-contact state where the close-contact load therebetween was almost zero when a load of 1N per driving wire 19 was applied in the axial direction, so as to set the wire to the close-contact state.

In FIG. 6, Comparative example (1N) is an example in which a load of 1N per driving wire was applied in the axial direction to a double coil spring in which an inner coil spring and an outer coil spring made of tension coils with no gap between the wires were screwed together. Comparative example (3N) is an example in which a load of 3N per driving wire was applied in the axial direction to a double coil spring in which an inner coil spring and an outer coil spring made of tension coils are screwed together.

These springs were attached to @5 forceps, a bending load was applied to displace the springs from the extended state to the flexed state, and the load was then removed to compare whether or not the springs returned to the original point of the extended state.

In FIG. 6, the vertical axis represents the displacement of the end effector, and the horizontal axis represents the bending angle of the flexural structure 1. Two lines are displayed for the same spring, one above the other, where the line above shows the change data from the extended state to the flexed state, and the line at the bottom shows the restoration data from the flexed state to the extended state.

As shown in FIG. 6(B), in Comparative example (1N) and Comparative example (3N), even when the bending load was removed, the inter-wire friction exceeded the restoring force when returning to the extended state, and the bending angle did not return to zero at the original point.

In contrast, in Example (1N) and Example (3N), the inter-wire friction fell below the restoring force until the extended state was reached, and the bending angle returned to zero at the original point.

Thus, in this example, by setting the close-contact state so that the friction between the wires is small to counteract the restoring force of the multi-structured flexible body 15 returning from the flexed state to the extended state, the multi-structured flexible body 15 in the flexed state may be reliably restored to the origin in the extended state.

Further, since the initial load may be ignored even during times other than the restoration, this contributes to the compression resistance of the double coil spring 16.

### Embodiment 2

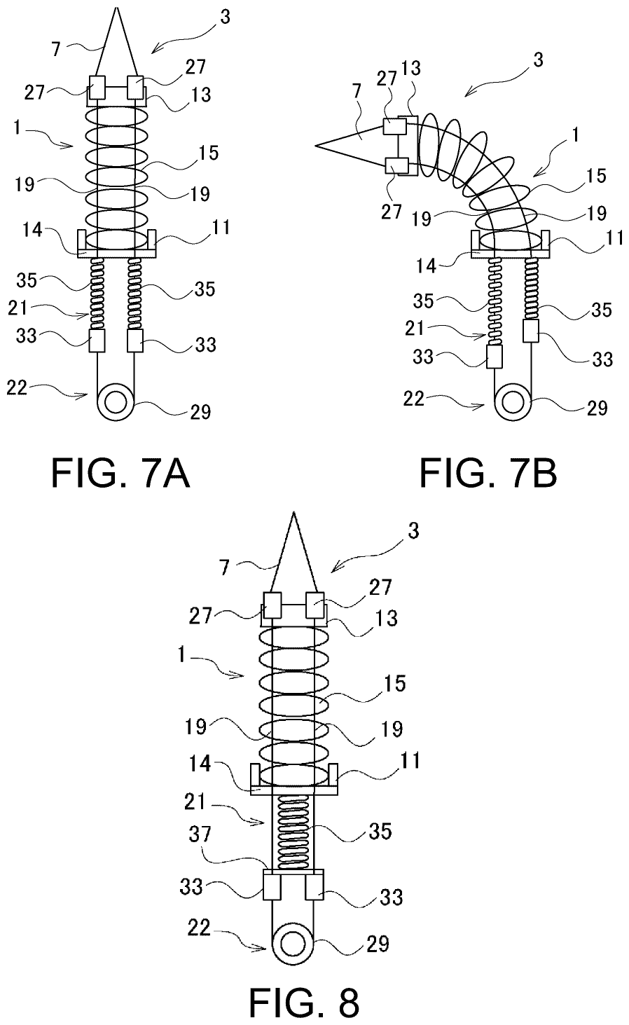

FIGS. 7(A) and (B) are conceptual diagrams of the manipulator to which the flexural structure according to Embodiment 2 of the present invention is applied, in which FIG. 7(A) shows the extended state, and FIG. 7(B) shows the flexed state. In Embodiment 2, the same reference numerals are used for configurations corresponding to those in Embodiment 1, and redundant description will be omitted.

The close-contact setting part 21 of Embodiment 2 includes receiving parts 33 provided on the other side of the driving wires 19 and compression coil springs 35 as elastic bodies interposed between the fixed receiving body 14 and the receiving parts 33. The receiving parts 33 are coupled to the driving wires 19 by crimping.

The compression coil spring 35 is provided so as to fit into each of the plurality of driving wires 19. Each compression coil spring 35 applies tension to the plurality of driving wires 19 with respect to the movable part 13 in the extended state of the multi-structured flexible body 15.

Thus, by setting the load of the compression coil springs 35, tension may be applied to the plurality of driving wires 19, and the close-contact state of the inner and outer coil springs 25 and 23 may be set.

It is noted that the compression coil spring 35 serving as the elastic body may be made of metal, resin, or the like, and may have an appropriate shape in accordance with the elastic modulus, etc. When the elastic body is made of rubber or the like, it may be in the form of a column or a tube.

The compression coil springs 35 are arranged in parallel with the driving wires 19 so as to exert elastic force in the axial direction. Here, “parallel” means that the compression coil springs 35 are arranged so that the axial direction and the direction in which the elastic force acts are parallel. However, the two directions do not need to be strictly parallel, and a case in which one of the directions is slightly inclined with respect to the other is also included in the term “parallel.”

Each compression coil spring 35 has a dimension in the axial direction in a free state that is set to be larger than the dimension in the axial direction between the receiving parts 33 and the attaching base part 11. Thus, each compression coil spring 35 is compressed in accordance with the dimensional difference between the receiving parts 33 and the attaching base part 11. This compression applies a load to each compression coil spring 35, and a tension corresponding to the load is applied to the driving wire 19.

Thus, in this Embodiment 2 as well, the close-contact state of the inner and outer coil springs 25 and 23 may be set by setting the load of the compression coil springs 35, and the same effects as those of Embodiment 1 may be obtained.

The operating force for compressing the compression coil springs 35 coaxial with the outer wire 19 may be assisted by the elastic force of the compression coil springs 35 coaxial with the inner wire 19 stretching. Thus, an increase in the overall operating force for bending the flexural structure 1 may be suppressed, and bending of the flexural structure 1 may be easily performed.

### Embodiment 3

FIG. 8 is a conceptual diagram showing the manipulator to which the flexural structure according to Embodiment 3 of the present invention is applied. In Embodiment 3, the same reference numerals are used for configurations corresponding to those in Embodiment 2, and redundant description will be omitted.

In the close-contact setting part 21 of Embodiment 3, a compression coil spring 21, which is a single elastic body, is used for the pair of driving wires 19 of the flexural structure 1. Specifically, a support member 37 is provided across the receiving parts 33 of the pair of driving wires 19, and the elastic body 21 is interposed between the support member 37 and the fixed receiving body 14. The rest is the same as Embodiment 2.

The support member 37 is a plate-like body provided across the receiving parts 33 of the pair of driving wires 19. The driving wires 19 are inserted through the support member 37. The support member 37 is pressed against the receiving parts 33 by the compression coil spring 21. The support member 37 may be integrally formed with the receiving parts 33.

In Embodiment 3, the same effect as Embodiment 2 may be achieved.

### Embodiment 4

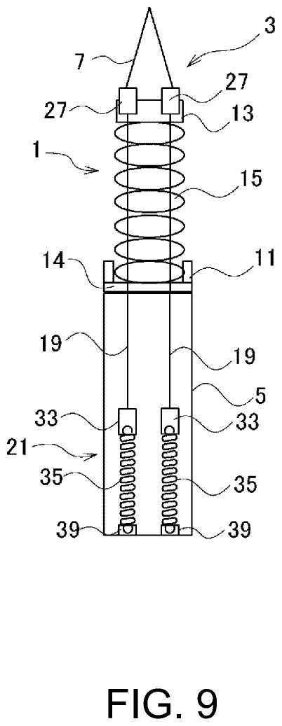

FIG. 9 is a conceptual diagram showing the manipulator to which the flexural structure according to Embodiment 4 of the present invention is applied. In Embodiment 4, the same reference numerals are used for configurations corresponding to those in Embodiment 1, and redundant description will be omitted.

In Embodiment 4, the elastic body is a tension coil spring 21, which is provided between the support part 39 of the shaft 5 and the receiving parts 33 of the driving wires 19. Specifically, the shaft 5 is provided with a support part 39 positioned opposite to the receiving part 33 of each driving wire 19 in the axial direction. Since the fixed receiving body 14 is fixed to the shaft 5, the support parts 39 are also components that the fixed receiving body 14 has. The tension coil spring 21 is interposed between the support parts 39 and the receiving parts 33.

In Embodiment 4, the pulley 29 is omitted, and the receiving parts 33 are coupled to the operating mechanism.

Thus, the tension of the driving wires 19 may be set by setting the load of the tension coil spring 21. This tension allows the setting of the close-contact state of the inner and outer coil springs 25 and 23, and the same effect as that of Embodiment 1 may be obtained.

However, a pulley 29 may be provided similarly to Embodiment 1. The rest is the same as Embodiment 1.

The support parts 39 may be provided at the end part of the shaft 5 or within the shaft 5. The shape of the support parts 39 may be any shape that may support the tension coil spring 21.

### REFERENCE SIGNS LIST

1 Flexural structure
5 Shaft
11 Attaching base part
13 Movable part (one-side receiving member)
14 Fixed receiving body (other-side receiving member)
15 Multi-structured flexible body
16 Double coil spring
17 Flexible tube
19 Driving wire (cord-like member)
21 Close-contact setting part
22 Operation part
23 Outer coil spring
25 Inner coil spring

1. A flexural structure, in which a multi-structured flexible body is bendable from an extended state to a flexed state, the flexural structure comprising:
an outer coil spring, constituting the multi-structured flexible body;
an inner coil spring, provided inside the outer coil spring and having a wire overlap a wire of the outer coil spring in a coil radial direction to constitute the multi-structured flexible body;
a one-side receiving member, receiving one side of the multi-structured flexible body; and
an other-side receiving member, receiving an other side of the multi-structured flexible body;
wherein a close-contact state between wires of the inner coil spring and the outer coil spring is set in an extended state of the multi-structured flexible body in accordance with a spacing between the one-side receiving member and the other-side receiving member.

2. The flexural structure according to claim 1, wherein
when setting the close-contact state, a friction between the wires is made smaller with respect to a restoring force of the multi-structured flexible body returning from the flexed state to the extended state.

3. The flexural structure according to claim 1, comprising:
a close-contact setting part, adjusting the spacing between the one-side receiving member and the other-side receiving member and setting a close-contact state between the wires of the inner coil spring and the outer coil spring.

4. The flexural structure according to claim 3, wherein the close-contact setting part comprises a plurality of cord-like members, one side of which is fixed to the one-side receiving member and an other side of which is routed through the other-side receiving member, for operating a displacement of the multi-structured flexible body to a flexed state, and
the cord-like members are shared when setting the close-contact state.

5. The flexural structure according to claim 4, wherein
the close-contact setting part comprises an operation part that is provided on the other side of the cord-like members and that operates a displacement between an extended state and a flexed state of the multi-structured flexible body by relative pulling and pulling back operations between the plurality of cord-like members,
the operation part is supported in a position adjustable manner toward a direction of the multi-structured flexible body in the extended state, and
the close-contact state is set by adjusting a position of the operation part in an extended state of the multi-structured flexible body.

6. The flexural structure according to claim 5, wherein
the operation part comprises a pulley that is supported by the other-side receiving member and is rotatably operable and movable for wrapping the cord-like members around the one-side receiving member, and
the close-contact setting part comprises a tensioner that adjusts a position of the pulley.

7. The flexural structure according to claim 4, wherein
the close-contact setting part comprises: a receiving part, provided on the other side of the cord-like members; and an elastic body, interposed between the other-side receiving member and the receiving part and applying tension to the plurality of cord-like members with respect to the one-side receiving member in an extended state of the multi-structured flexible body, and
the close-contact state is set by applying the tension by the elastic body.

8. The flexural structure according to claim 2, comprising:
a flexible tube, having one end part coupled to the one-side receiving member, enclosing the inner coil spring and the outer coil spring, and constituting the multi-structured flexible body along with the inner coil spring and the outer coil spring; and
an attaching base part, coupled to an other end part of the flexible tube and penetrating the inner coil spring and the outer coil spring along with the flexible tube,
wherein the other-side receiving member is fixed in an end part of a hollow shaft, and
the close-contact setting part sets the close-contact state by coupling the attaching base part to the end part of the hollow shaft to move the attaching base part and having the other-side receiving member to receive other end parts of the inner coil spring and the outer coil spring.

9. A half-finished product of a flexural structure in which a multi-structured flexible body is bendable from an extended state to a flexed state, the half-finished product of the flexural structure comprising:
an outer coil spring, constituting the multi-structured flexible body;
an inner coil spring, provided inside the outer coil spring and having a wire overlap a wire of the outer coil spring in a non-close-contact state in a coil radial direction to constitute the multi-structured flexible body; and
a one-side receiving member, receiving one side of the multi-structured flexible body,
wherein when assembled to a device side, a close-contact state between wires of the inner coil spring and the outer coil spring is set in an extended state of the multi-structured flexible body in accordance with a spacing between the one-side receiving member and an other-side receiving member on the device side.
